(19) United States
(12) Patent Application Publication
(10) Pub. No.: US 2003/0229910 A1
(43) Pub. Date: Dec. 11, 2003

(54) Short fragment homologous replacement to provide BSE resistant cattle

(76) Inventors: Richard Metz, Lawrenceville, NJ (US); R. Michael Blaese, New Hope, PA (US)

Correspondence Address: ROPES & GRAY LLP, ONE INTERNATIONAL PLACE, BOSTON, MA, 02110-2624, US

(21) Appl. No.: 10/417,964

(22) Filed: Apr. 17, 2003

Related U.S. Application Data
(60) Provisional application No. 60/373,149, filed on Apr. 17, 2002.

(51) Int. Cl. A01K 67/027; A61K 48/00

(52) U.S. Cl. 800/15; 424/93.21

(57) ABSTRACT

A method for generating cattle resistant to Bovine Spongioform Encephalopathy through targeted alterations in the PrP gene is disclosed. The PrP gene of a cultured cells is altered to prevent its translation or to encode a dominant disease-resistant form of the protein, and the nucleus of the altered cell is used to clone a founder animal. In one embodiment, a single-stranded DNA fragment containing the alteration is used in single-stranded short fragment homologous replacement to alter the PrP gene.

### CROSS-REFERENCE TO RELATED APPLICATIONS

This application claims priority from U.S. Provisional Application No. 60/373,149, filed Apr. 17, 2002, the disclosure of which is incorporated by reference herein.

### BACKGROUND OF THE INVENTION

The modification of the genome of a cell can, in principle, be accomplished either by introducing a complete gene into the genome at a random position or by making a specific alteration in an existing, naturally occurring gene. In mammalian cells, endogenous enzymes that effect homologous recombination have been used to introduce disruptions in specific genes for more than a decade. The technique is termed homologous-recombination dependent gene targeting (hrdGT). Doetschman, T., et al., 1987, Nature 330, 576-78; Thomas K. R. & Capecchi, M. R., 1987, Cell 51, 503-12. These efforts involve the introduction of large pieces (several kilobases (kb)) of duplex DNA into the cell in the presence of a genetic selection system that distinguishes between homologous recombination and random insertion.

The use of an alternative has been described in mammalian cells. The technique is termed single-stranded short fragment homologous replacement (ssSFHR). A DNA fragment of intermediate size, typically 400 to 800 bp, is manufactured by excision from a plasmid vector or, alternatively, synthesized by PCR from a template. The short fragment is denatured by heat and the complementary strands can be optionally purified from each other. The technology is described in U.S. Pat. No. 6,010,908 by D.C. Gruenert, and in the scientific literature. Kapsa, R., et al., 2001, Human Gene Therapy 12, 629-42 (repair of murine dystrophin, unseparated strands); Colosima, A., et al., 2001, Mol. Therapy Vol. 3, No. 3 (episomal DNA in mammalian cells, unseparated strands); Goncz, K. K., et al., 1998, Hum. Mol. Genetics 7, 1913-19 (human cystic fibrosis transmembrane conductance regulator (CFTR), unseparated strands); Kunzelman, K., et al., 1996, Gene Therapy 3, 859-867 (murine CFTR, unseparated strands). The ssSFHR technique differs from hrdGT in several respects. The nucleic acid is shorter (400-800 nt) compared to several kb for hrdGT; in ssSFHR, the exogenous polynucleotide is denatured, i.e., single stranded, but is homologous with the target gene except for a few mutator nucleotides; in hrdGT, foreign genes are embedded in the exogenous nucleic acid; and, in hrdGT, a selection system is employed that distinguishes between homologous and illegitimate recombination, where in ssSFHR no such selection is required because illegitimate recombination does not occur at rates comparable to that of homologous recombination.

The present invention concerns the use of ssSFHR to modify the genome of cattle so that they are resistant to Bovine Spongioform Encephalopathy (BSE). BSE is a type of the so-called transmissible spongioform encephalopathies (TSE), which include ovine scrapie and human Creutzfeldt-Jakob Disease (CJD), as well as other diseases. A recent epidemic of over 170,000 cases of BSE occurred in the United Kingdom, which resulted in transmission of at least 130 cases to humans. The epidemic is believed to have been caused by the use of scrapie-infected sheep in the preparation of processed animal feed for the cattle, a process that was discontinued in 1988 and resulted in the reduction of the numbers of cases. Pattison, J., 1998, Emerg Infect. Dis. 4, 390-4; Nathanson, N., et al., Am. J Epidemiol. 45, 959-69.

TSE are the unique infectious diseases that are not transmitted by a nucleic acid-based disease organism. Rather, TSE result from the abnormal conformation of a brain protein, the prion protein (PrP). Prusiner, S. B., 1991, Science 252, 1515-22. The pathologic conformation consists of a 142-amino acid fragment of the PrP that adopts a predominantly β-pleated sheet conformation, which form catalyzes the conversion of other PrP to assume the pathological conformation. Peretz, D., 2001, Protein Science 10, 854-63; Wadsworth, J. D., et al., 1999, Curr Opin Genet Dev 9, 338-45. Though the hypothesis that TSE results from an infectious conformational change in the PrP is not universally accepted outside of the English-speaking world (see, e.g., Lasmezas, C.I., et al., 1997, Science 275, 402-5), it is widely accepted and has been confirmed by examples of inherited protein conformation in yeast. Lindquist, S., 1996, Mol. Psychiatry 1, 376-9; Lindquist, S., 1997, Cell 89, 495-8.

Whatever uncertainty may remain about the etiology of TSE, the ablation of the host PrP gene results in an animal that is resistant to the disease. Prusiner, S. B., et al., 1993, PNAS 90, 10608-12; Weissmann, C., & Aguzzi, A., 1999, Science 286, 914-15. In addition, dominant disease-resistant alleles of PrP having amino acid substitution can confer resistance to the disease as heterozygotes. Perrier, V. et al., 2002, PNAS 99, 13079-84. While certain biochemical and morphological abnormalities are associated with the PrP-ablated condition, the animals develop normally and appear healthy. Miele, G., et al., 2002, BBRC 291, 372-77; White, A. R., et al., 1999, Am J. Path. 155, 1723-30.

### SUMMARY OF THE INVENTION

The invention provides a method of rendering cattle resistant to BSE by ablation of the bovine PrP gene. Fragments of bovine PrP gene are cloned into bacteria and mutated by known techniques of site directed mutagenesis. The mutated cloned gene is used as a template to generate a short fragment (henceforth “SF”) of between 200-1000 bp, preferably between 400 and 800 bp using conventional oligonucleotide primed polymerase chain reaction amplification. There can be more than one genetic alteration encoded in an SF, but the alterations should be limited in size and extent, so that not more than four consecutive nucleotides of the SF will not be homologous to the target gene. A second alteration without physiological effects may be introduced to facilitate the subsequent isolation of mutant cells that have homologously recombined the SF. The differences between the sequence of the SF and that of the target gene (the “heterologies”) can either be mismatches, insertions or deletions. Ablation is caused by insertion of a frame shift mutation or multiple stop codons.

The SF is converted to single strand SF (“ssSF”), and then into a strand separated form (“s⁴SF”). The sequence of the SF will preferably be examined to determine self-complementary sequences that will cause extensive self-complementary secondary structure.

Once formed, the s⁴SF can be introduced into a somatic cell, typically a fibroblast, so as to induce ablation of the PrP gene. Selection of mutated clones is performed by cloning and PCR screening. To facilitate PCR screening, it is preferred that the ablating mutation create a readily observable restriction site, so that mutant clones can be identified without sequencing. Cattle incorporating the mutated PrP gene can be recovered by nuclear transfer to oocytes, using known techniques.

### DETAILED DESCRIPTION OF THE INVENTIONS

Preparation of the s⁴SF and generation of a mutant PrP gene. In one embodiment the invention consists of the use of a short fragment (SF) of single stranded DNA of between 200 and 1000 nt and, more preferably between 400 and 800 nt that is homologous (identical) with a fragment of the bovine PrP gene, except at a limited number of positions, typically fewer than 10, which are designed to introduce ablating mutations into the PrP gene and, optionally generate an additional alteration to facilitate identification of the modified PrP locus by a combination of allele-specific PCR and a secondary detection. The sequence of the bovine PrP gene is found at GENBANK accession No. AJ298878, the sequence of the PrP cDNA can be found as accession AB001468, which are hereby incorporated by reference.

Construction of the desired, mutated sequence can be most readily accomplished by in vitro site-directed mutagenesis. The techniques involved are well known in the art. Perrin, S., & Gilliland, G., 1990, Nucleic Acid Research 18, 7433; Landt, O., et al., 1990, Gene 96, 125-8; Nassal M., & Rieger, A., 1989, Nucleic Acids Research 18, 3077-8; Hemsley, A., et al., 1989, Nucleic Acids Research 17, 6545-51. Implementation of these techniques require that the target gene or a fragment of the gene that encompasses the sequenced to be modified is available in recombinant clones. Having constructed the appropriate desired sequence, the SF itself can be synthesized by routine polymerase chain reaction (“PCR”). When s⁴SF are to be used, the synthesis employs one 5′-biotinylated primer and one underivitized primer. The strands are separated as described below. The synthesis of 5′-biotinylated primers is well known. Cook, A. F., et al., 1988, Nucleic Acids Research 16, 4077-95; Connolly, B. A., 1988, Nucleic Acids Research 15, 3131-9.

After the SF is synthesized in a duplex form, i.e., the form in which the fragment is Watson-Crick bound to its complement, a single stranded SF can be prepared. The preparation is most simply accomplished by heat denaturation (heating to 95° C.) followed by rapid cooling to 4° C. This process results in a mixture of strands of both polarity having no or essentially no intermolecular Watson-Crick base pairings. However, continued incubation of the mixture at elevated temperatures can result in the formation of inter-molecular Watson-Crick pairings.

The separation of the complementary strands can be readily accomplished when one of the two primers used in the PCR synthesis of the SF is biotinylated. Separation of the product can be effected by binding the biotinylated strand to immobilized avidin as follows:

Double stranded SF (ds-SF) products can be prepared by PCR using two primers, one of which contained a biotin at the 5′ end.

Single strand preparation:

Single strands were generated by binding the biotinylated PCR product to avidin-magnetic beads (Dyonex).

The displaced strand (D-ssSF not containing biotin) was isolated by denaturing the bound dsPCR fragment under high pH (0.5 M NaOH) 1-2 minutes.

The “displaced strand” (supernatant) was removed from the beads using a magnet or centrifugation, neutralized with acid (27 ul cHCL per 500 :1 0.5 M NaOH) and dialyzed (1000×volume 0.1 M Tris pH 7.0, then 2 Times 1000×volume of water). The displaced strand was then concentrated by ethanol precipitation or spin concentrators.

The immobilized strand (B-SF) attached to the beads was neutralized with 2 Tris 0.1 M pH 7.0 washes followed by 2 water washes. The immobilized strand was removed from the magnetic beads in water following heat treatment (95° C.).

Both displaced and immobilized strands individually have activity.

Typically the displaced strand was more active. Either the coding or non-coding strand may be used to introduce the modification into the targeted gene.

The s⁴SF can be introduced into a bovine cell, such as a bovine fibroblast, by any method that can be used to introduce duplex DNA into the cell. The preferred method is by microinjection, which allows for individual inoculation of pre-selected, adherent cells in an controlled manner.

A cell containing the modified target gene can be isolated by cloning and PCR testing of the cloned cells prior to the regeneration of whole animals. Several methods can be employed to identify a clone of cells in which the SF has altered the PrP locus.

A particular method, which is suitable when the frequency of altered cells is low is termed “coupled detection” (CD) and was described in commonly owned U.S. patent application Ser. No. 10/298,859, filed Nov. 18, 2002, by R. Metz which is hereby incorporated by reference in its entirety. In CD, two alternations, typically between 50 and 100 nucleotides apart, are introduced using a single SF. After a population containing a putatively modified cell is obtained; the population is divided into replicate subgroups. A PCR reaction is performed on genomic DNA from a replicate of each subgroup using a PCR primers that will amplify the target sequence but not the SF. The products of this reaction are diluted and used as template for the second PCR reaction. The second PCR reaction is performed using a PCR primer that preferentially anneals to the sequence of one of the alterations compared to the wild-type sequence and a non-selective second primer. The PCR reaction is designed so that the product includes the site of the second alteration. Suitable selection of annealing temperature results in the preferential amplification of the altered fragment relative to the wild-type. The preferential amplification permits the ready detection of a single copy of the altered genotype in a subgroup of several thousand by detection of the second alteration in the PCR product. A second alteration that creates or deletes a restriction enzyme recognition such that the presence of a mutant locus can be detected by a restriction enzyme digest is particularly preferred because of the ease in detection.

Using CD, a large population of cells can be readily screened to detect a rare cell that contains the linked alterations. The screening is performed by subdividing the populations into subgroups or about 5,000. The replicates from subgroups that contain a single copy of the rare modified cell are further subdivided and cultured. Successive cycles of subdivision, replica formation and detection can be used to isolate the rare modified cell from the population.

In a less preferred method, a PCR primer that preferentially anneals to the mutant sequence compared to the wild-type sequence is used to PCR-amplify a genomic fragment from a population of cells under such temperature conditions that only the mutant sequence, if present, yields a product.

In an alternative embodiment, the alteration in the bovine PrP gene can be made to increase the relative stability of the soluble form of the PrP protein. Mutations that increase the stability have been identified by comparison of the susceptibility of different strains of sheep to scrapie with polymorphism in the ovine PrP gene. Mutations at positions 136, 154 and 171 were found to be protective. Drogenmuller, C., et al., 2001, Vet. Res. 149, 349-52. The same mutations can be introduced into the bovine PrP gene in an alternative embodiment of the invention.

In yet another embodiment, an alteration of the bovine PrP gene by mutations that confer a dominant disease-resistant phenotype can be introduced into the bovine PrP gene, so that animals would not need to be homozygous for the altered Prp gene to be resistant to the disease.

Generation of BSE resistant cattle: The generation of domestic animals containing site-specific mutations has been made possible by recent advances in nuclear transplantation from somatic cells into a competent germ-line cell, i.e., oocytes or cells of a blastocyst (blastomers). These techniques are referred to in general as “cloning” or “animal cloning” because they enable the practitioner to make a genetically identical individual from an explanted somatic cell. The techniques are described in detail in U.S. Pat. No. 6,147,276 and No. 6,252,133.

Scientific publications describing the technology teach that with some species-specific adaptations the techniques have proved successful in sheep, cattle and swine. Schnieke, A. E., et al., 1997, Science 278, 2130-33; Wilmut, I., et al., 1997, Nature 385, 810-3; Polejaeva, I. A., et al., 2000, Nature 407, 29-30. A current review of the field can be found in Kuhholtzer, B., & Prather, R. S., 2000, Proc. Soc. Exp. Med. 224, 240-45.

It is expected that the originally mutated somatic nucleus will be heterozygous for the PrP mutation. Accordingly, the generation of BSE resistant stock will require interbreeding the founder stock in order to isolate the mutation in homozygous form when the alteration of the PrP gene is designed to prevent its translation. The presence of the modified PrP in offspring of parents carrying a PrP disease-resistant allele can be determined through a DNA-based assay which may include techniques commonly known in the art such as RFLP mapping, SNP detection, southern blots, PCR amplification and direct sequencing. As an alternative to generating an animal homozygous for alterations in the PrP gene, cell lines prepared from embryos derived in the first round of nuclear transfer cloning can be retargeted by SFHR to alter the second PrP allele. The alteration of the second allele can be the same as that of the first allele or alternatively it can be different to aid in the identification of cells having both PrP alleles modified. These mutant cell lines homozygous for altered PrP alleles heterozygous can be used to redone an animal homozygous for the desired PrP gene mutation.

DNA fragments for SFHR are synthesized by PCR in a two step process using a commercially available vector into which exon 3 of bovine PrP has been inserted. Two types of primers are used. A mutational primer is used to alter the PiP sequence in the vector.

After the mutation is introduced production primers are used to make the SFHR duplex DNA by PCR using the mutated vector as template.

Listed below of Forward and Reverse production primers (FP and RP) and mutational primers which are labeled according to the position of the mutation in the amino acid sequence.

| | |
|---|---|
| SFHR PCR Primers | |
| LOCATION | NUCLEOTIDE |

| | | | |
|---|---|---|---|
| FP5 | 5′ GTGGCCATGTGGAGTGA | (SEQ ID NO:1) | 281 |
| RP5 | 5′ CCCAACCTGGTAAAGATTAAG | (SEQ ID NO:2) | 1061 |
| | (rc-cttaatctttaccaggttggg) | (SEQ ID NO:3) | |
| FP4 | 5′ CTGTTTATAGCTGATGCCACT | (SEQ ID NO:4) | 130 |
| RP4 | 5′ ACGGTTGCCTCCAGGAC | (SEQ ID NO:5) | 375 |
| | (rc-gtcctggaggcaaccgt) | (SEQ ID NO:6) | |
| RP3 | 5′ GGCTTACTGGGTTTGTTCC | (SEQ ID NO:7) | 567 |
| | (rc-ggaacaaacccagtaagcc) | (SEQ ID NO:8) | |
| RP2 | 5′ GGCCTGTAGTACACTTGGTTG | (SEQ ID NO:9) | 745 |
| | (rc-caaccaagtgtactacaggcc) | (SEQ ID NO: 10) | |

Mutagenic primers and SFHR combinations

| | | | |
|---|---|---|---|
| Prp-mutant | | | |
| Primer set | Name | Sequence | SFHR |

| | | | | |
|---|---|---|---|---|
| Prp-0-1 | W9stop 5′ CACATAGGCAGTTAGATCCTGGTTCTC3′ | (SEQ ID NO:11) | FP4/Rp2, | |
| | | | | FP4/RP3, |
| | | | | FP4/RP4, |
| | | | | FP4/RP5 |
| Prp-0-2 | W18stop | 5′ TTTGTGGCCATGTAGAGTGACGTGGGC3′ | (SEQ ID NO:12) | FP4/Rp2, |
| | | | | FP4/RP3, |
| | | | | FP4/RP4, |
| | | | | FP4/RP5 |
| Prp-0-3 | C24stop | 5′ GACGTGGGCCTCTGAAAGAAGCGACCA3′ | (SEQ ID NO:13) | FP4/Rp2, |
| | | | | FP4/RP3, |
| | | | | FP4/RP4, |
| | | | | FP4/RP5 |
| Prp-0-4 | K3stop | 5′ GTCATCATGGTGTAAAGCCACATAGGC3′ | (SEQ ID NO:14) | FP4/Rp2, |
| | | | | FP4/RP3, |
| | | | | FP4/RP4, |
| | | | | FP4/RP5 |
| Prp-0-d5 | V2del | 5′ GTCATCATGGT:AAAAGCCACATAGGC3′ | (SEQ ID NO:15) | FP4/Rp2, |
| | | | | FP4/RP3, |
| | | | | FP4/RP4, |
| | | | | FP4/RP5 |
| Prp-0-d6 | H5del | 5′ GGTGAAAAGCCA:ATAGGCAGTTGGAT3′ | (SEQ ID NO:16) | FP4/Rp2, |
| | | | | FP4/RP3, |
| | | | | FP4/RP4, |
| | | | | FP4/RP5 |
| Prp-ARR | Q178R | 5′ AGGCCAGTGGATCGGTATAGTAACCAG3′ | (SEQ ID NO:17) | FP4/RP5, |
| | | | | FP4/RP3, |
| | | | | FP4/RP4, |
| | | | | FP4/RP5 |

Example: The approach for generating animals resistant to Transmissible Spongiform Encephalopathy (TSE) or Bovine Spongiform Encephalopathy (BSE) will proceed in two parallel tracks. A non-functional PrP allele will be generated in a bovine primary cell line using SFHR (GenEdit) molecules (PrP0-1 and/or PrP0-2, other molecules disrupting the open reading frame may also be attempted such as PrP-0-3, -4, -5, -6 etc). In addition, mutagenic PCR primers will be used to insert a point mutation in a restriction enzyme recognition sequence within 100 nucleotides of any of the above mutations. Mutant cells will be generated which have incorporated the mutant sequence by homologous recombination, and clones of these cells will be screened for the presence of the mutant sequences. Replicate subcultures will be generated and DNA prepared for PCR-amplification. In order to increase our sensitivity and selectivity two rounds of PCR amplification will be performed. The first reaction will use a primer set flanking the PrP targeted region. The products from the first round reaction will be diluted 10,000 fold and used as a template for a an allele-enrichment PCR reaction, where one of the primers is designed to preferentially bind the mutant sequence to selectively enrich for sequences containing the PrP mutations. The allele-enriched PCR product will then be digested with the restriction enzyme whose recognition site was mutated. Uncut PCR products are those that contain the mutant sequences, whereas the presence of two fragments will represent the presence of the wildtype PrP. Subcultures containing the mutant form of PrP will be further subdivided and the process of screening for the mutant PrP will be reiterated until a pure subculture containing modified mutant cells is isolated.

From the modified cell line animals will be generated using nuclear transfer technology. The reduction of a functional PrP allele may have protective properties based on reduced gene product. A homozygous PrP-0 animal (PrP-0/PrP-0) can be generated by back crossing PrP-0 heterozygotes. In a similar fashion, a BSE resistant allele will be introduced into a breeding stock using SFHR molecules (bPrPAAR and/or other molecules affecting resistant phenotype) to introduce a polymorphism barrier to TSE (BSE). The current evidence for polymorphism barriers to TSE (Scrapie) has been described for scrapie resistant herds of sheep containing Alanine (A), Arginine (R) and Arginine (R) at codons 136, 154, and 171, respectively. The bovine sequence contains the resistance-associated amino acids at positions homologous to 136 and 154 and only the amino acid at 171 need be modified. Homozygote PrPARR/PrPAAR or PrPARR/PrP-0 animals can be generated using standard back crossing and cross breeding strategies with the appropriate homozygote/heterozygote animals.

A number of mutations that can be generated including several null alleles. Below are examples of three nonsense and two frame shift null mutations. Also given are the base substitutions that generate a bovine PrP-ARR. SFHR molecules will be single stranded coding or non-coding, or denatured double stranded. All null generating SFHR molecules will extend into intron 2 and terminate in the exon 3 (Coding region of PRP). The PrP-ARR alelle will need an SFHR molecule whose sequences are contained in exon 3.

| | | | |
|---|---|---|---|
| bovine ex. | #-237 | cta gga aac aga gcc agg aat tat ttt aag gtc | |
| bovine ex. | #-204 | aac ttt gtc ctt aga gaa gga aga gtt gtg tta | |
| bovine ex. | #-171 | aca ctt tac cta taa tta ctt tcg tga gat gta | |
| bovine ex. | #-138 | tgg aat gtg aag aat att tat gac cta gac tgt | |
| bovine ex. | #-105 | tta tag ctg atg cca ctg cta tgc agt cat tat | |
| bovine ex. | #-72 | gct aca gac ttt aag tga ttt tta cat ggg cat | |
| bovine ex. | #-39 | atg atg ctg aca ccc tct tta ttt tgc agA TAA | |
| bovine ex. | #-6 | GTC ATC ATG GTG AAA AGC CAC ATA GGC AGT TGG | (SEQ ID NOS:18-19) |
| | | M V K S H I G S W | |
| PrP-0-1 | #-6 | GTC ATC ATG GTG AAA AGC CAC ATA GGC AGT TAG | (SEQ ID NOS:20-21) |
| | | M V K S H I G S * | |
| PrP-0-2 | #-6 | GTC ATC ATG GTG AAA AGC CAC ATA GGC AGT TGG | (SEQ ID NOS:22-23) |
| | | M V K S H I C S W | |
| PrP-0-3 | #-6 | GTC ATC ATG GTG AAA AGC CAC ATA GGC AGT TGG | (SEQ ID NOS:24-25) |
| | | M V K S H I G S W | |
| Prp-0-4 | #-6 | GTC ATC ATG GTG TAA AGC CAC ATA GGC AGT TGG | (SEQ ID NO:26) |
| | | M V * | |
| PrP-0-d5 | #-6 | GTC ATC ATG GT: AAA AGC CAC ATA GGC AGT TGG | (SEQ ID NOS:27- 28) |
| | | M V K A T * | |
| PrP-0-d6 | #-6 | GTC ATC ATG GTG AAA AGC CA: ATA GGC AGT TGG | (SEQ ID NOS:29-30) |
| | | M V K S Q * | |
| PrP-ARR | #-6 | GTC ATC ATG GTG AAA AGC CAC ATA GGC AGT TGG | (SEQ ID NOS:31-32) |
| | | M V K S H I G S W | |
| bovine ex. | #28 | ATC CTG GTT CTC TTT GTG GCC ATG TGG AGT GAC | |
| | | I L V L F V A M W S D | |
| PrP-0-1 | #28 | ATC CTG GTT CTC TTT GTG GCC ATG TGG AGT GAC | |
| PrP-0-2 | #28 | ATC CTG GTT CTC TTT GTG GCC ATG TAG AGT GAC | |
| | | I L V L F V A M * | |
| PrP-0-3 | #28 | ATC CTG GTT CTC TTT GTG GCC ATG TGG AGT GAC | |
| | | I L V L F V A M W S D | |
| Prp-0-4 | #28 | ATC CTG GTT CTC TTT GTG GCC ATG TGG AGT GAC | |
| PrP-0-d5 | #28 | ATC CTG GTT CTC TTT GTG GCC ATG TGG AGT GAC | |
| PrP-0-d6 | #28 | ATC CTG GTT CTC TTT GTG GCC ATG TGG AGT GAC | |
| PrP-ARR | #28 | ATC CTG GTT CTC TTT GTG GCC ATG TGG AGT GAC | |
| | | I L V L F V A M W S D | |
| bovine ex. | #61 | GTG GGC CTC TGC AAG AAG CGA CCA AAA CCT GGA | |
| | | V G L C K K R P K P G | |
| PrP-0-1 | #61 | GTG GGC CTC TGC AAG AAG CGA CCA AAA CCT GGA | |
| PrP-0-2 | #61 | GTG GGC CTC TGC AAG AAG CGA CCA AAA CCT GGA | |
| PrP-0-3 | #61 | GTG GGC CTC TGA AAG AAG CGA CCA AAA CCT GGA | |
| | | V G L * | |
| Prp-0-4 | #61 | GTG GGC CTC TGC AAG AAG CGA CCA AAA CCT GGA | |
| PrP-0-d5 | #61 | GTG GGC CTC TGC AAG AAG CGA CCA AAA CCT GGA | |
| PrP-0-d6 | #61 | GTG GGC CTC TGC AAG AAG CGA CCA AAA CCT GGA | |
| PrP-ARR | #61 | GTG GGC CTC TGC AAG AAG CGA CCA AAA CCT GGA | |
| | | V G L C K K R P K P G | |
| bovine ex. | #94 | GGA GGA TGG AAC ACT GGG GGG AGC CGA TAC CCA | |
| | | G G W N T G G S R Y P | |
| PrP-0-1 | #94 | GGA GGA TGG AAC ACT GGG GGG AGC CGA TAC CCA | |
| PrP-0-2 | #94 | GGA GGA TGC AAC ACT GGG GGG AGC CGA TAC CCA | |
| PrP-0-3 | #94 | GGA GGA TGG AAC ACT GGG GGG AGC CGA TAC CCA | |
| Prp-0-4 | #94 | GGA GGA TGG AAC ACT GGG CGG AGC CGA TAC CCA | |
| PrP-0-d5 | #94 | GGA GGA TGG AAC ACT GGG GGG AGC CGA TAC CCA | |
| PrP-0-d6 | #94 | GGA GGA TGG AAC ACT GGG GGG AGC CGA TAC CCA | |
| PrP-ARR | #94 | GGA GGA TGG AAC ACT GGG GGG AGC CGA TAC CCA | |
| | | G C W N T C G S R Y P | |
| bovine ex. | #127 | GGA CAG GGC AGT CCT GGA GGC AAC CGT TAT CCA | |
| | | G Q G S P C G N R Y P | |
| PrP-0-1 | #127 | GGA CAG GGC AGT CCT GGA GGC AAC CGT TAT CCA | |
| PrP-0-2 | #127 | GGA CAG GGC AGT CCT GGA GGC AAC CGT TAT CCA | |
| PrP-0-3 | #127 | GGA CAG GGC AGT CCT GGA GGC AAC CGT TAT CCA | |
| Prp-0-4 | #127 | GGA CAG GGC AGT CCT GGA GGC AAC CGT TAT CCA | |
| PrP-0-d5 | #127 | GGA CAG GGC AGT CCT GGA GGC AAC CGT TAT CCA | |
| PrP-0-d6 | #127 | GGA CAG GGC AGT CCT GGA GGC AAC CGT TAT CCA | |
| PrP-ARR | #127 | GGA CAG GGC AGT CCT GGA GGC AAC CGT TAT CCA | |
| | | G Q G S P G G N R Y P | |
| bovine ex. | #160 | CCT CAG GGA GGG GGT GGC TGG GGT CAG CCC CAT | |
| | | P Q G G G G W G Q P H | |
| PrP-0-1 | #160 | CCT CAG GGA GGG GGT GGC TGG GGT CAG CCC CAT | |
| PrP-0-2 | #160 | CCT CAG GGA GGG GGT GGC TGG GGT CAG CCC CAT | |
| PrP-0-3 | #160 | CCT CAG GGA GGG GGT GGC TGG GGT CAG CCC CAT | |
| Prp-0-4 | #160 | CCT CAG GGA GGG GGT GGC TGG GGT CAG CCC CAT | |
| PrP-0-d5 | #160 | CCT CAG GGA GGG GGT GGC TGG GGT CAG CCC CAT | |
| PrP-0-d6 | #160 | CCT CAG GGA GGG GGT GGC TGG GGT CAG CCC CAT | |
| PrP-ARR | #160 | CCT CAG GGA GGG GGT GGC TGG GGT CAG CCC CAT | |
| | | P Q G G G G W G Q P H | |
| bovine ex. | #193 | GGA GGT GGC TGG GGC CAG CCT CAT GGA GGT GGC | |
| | | G G G W G Q P H G G G | |
| PrP-0-1 | #193 | GGA GGT GGC TGG GGC CAG CCT CAT GGA GGT GGC | |
| PrP-0-2 | #193 | GGA GGT GGC TGG GGC CAG CCT CAT GGA GGT GGC | |
| PrP-0-3 | #193 | GGA GGT GGC TGG GGC CAG CCT CAT GGA GGT GGC | |
| Prp-0-4 | #193 | GGA GGT GGC TGG GGC CAG CCT CAT GGA GGT GGC | |
| PrP-0-d5 | #193 | GGA GGT GGC TGG GGC CAG CCT CAT GGA GGT GGC | |
| PrP-0-d6 | #193 | GGA GGT GGC TGG GGC CAG CCT CAT GGA GGT GGC | |
| PrP-ARR | #193 | GGA GGT GGC TGG GGC CAG CCT CAT GGA GGT GGC | |
| | | G G G W G Q P H G G G | |
| bovine ex. | #226 | TGG GGC CAG CCT CAT GGA GGT GGC TGG GGT CAG | |
| | | W G Q P H G G G W G Q | |
| PrP-0-1 | #226 | TGG GGC CAG CCT CAT GGA GGT GGC TGG GGT CAG | |
| PrP-0-2 | #226 | TGG GGC CAG CCT CAT GGA GGT GGC TGG GGT CAG | |
| PrP-0-3 | #226 | TGG GGC CAG CCT CAT GGA GGT GGC TGG GGT CAG | |
| Prp-0-4 | #226 | TGG GGC CAG CCT CAT GGA GGT GGC TGG GGT CAG | |
| PrP-0-d5 | #226 | TGG GGC CAG CCT CAT GGA GGT GGC TGG GGT CAG | |
| PrP-0-d6 | #226 | TGG GGC CAG CCT CAT GGA GGT GGC TGG GGT CAG | |
| PrP-ARR | #226 | TGG GGC CAG CCT CAT GGA GGT GGC TGG GGT CAG | |
| | | W C Q P H G G G W G Q | |
| bovine ex. | #259 | CCC CAT GGT GGT GGC GGC GGA CAC CCA CAT GGT | |
| | | P H G G G W G Q P H G | |
| PrP-0-1 | #259 | CCC CAT GGT GGT GGC TGG GGA CAG CCA CAT GGT | |
| PrP-0-2 | #259 | CCC CAT GGT GGT GGC TGG GGA CAG CCA CAT GGT | |
| PrP-0-3 | #259 | CCC CAT GGT GGT GGC TGG GGA CAG CCA CAT GGT | |
| Prp-0-4 | #259 | CCC CAT GGT GGT GGC TGG GGA CAG CCA CAT GGT | |
| PrP-0-d5 | #259 | CCC CAT GGT GGT GGC TGG GGA CAG CCA CAT GGT | |
| PrP-0-d6 | #259 | CCC CAT GGT GGT GGC TGG GGA CAG CCA CAT GGT | |
| PrP-ARR | #259 | CCC CAT GGT GGT GGC TGG GGA CAG CCA CAT GGT | |
| | | P H G G G W G Q P H C | |
| bovine ex. | #292 | GGT GGA GGC TGG GGT CAA GGT GGT ACC CAC GGT | |
| | | G G G W G Q G G T H G | |
| PrP-0-1 | #292 | GGT GGA GGC TGG GGT CAA GGT GGT ACC CAC GGT | |
| PrP-0-2 | #292 | GGT GGA GGC TGG GGT CAA GGT GGT ACC CAC GGT | |
| PrP-0-3 | #292 | GGT GGA GGC TGG GGT CAA GGT GGT ACC CAC GGT | |
| Prp-0-4 | #292 | GGT GGA GGC TGG GGT CAA GGT GGT ACC CAC GGT | |
| PrP-0-d5 | #292 | GGT GGA GGC TGG GGT CAA GGT GGT ACC CAC GGT | |
| PrP-0-d6 | #292 | GGT GGA GGC TGG GGT CAA GGT GGT ACC CAC GGT | |
| PrP-ARR | #292 | GGT GGA GGC TGG GGT CAA GGT GGT ACC CAC GGT | |
| | | G G G W G Q G G T H G | |
| bovine ex. | #325 | CAA TGG AAC AAA CCC AGT AAG CCA AAA ACC AAC | |
| | | Q W N K P S K P K T N | |
| PrP-0-1 | #325 | CAA TGG AAC AAA CCC AGT AAG CCA AAA ACC AAC | |
| PrP-0-2 | #325 | CAA TGG AAC AAA CCC AGT AAG CCA AAA ACC AAC | |
| PrP-0-3 | #325 | CAA TGG AAC AAA CCC AGT AAG CCA AAA ACC AAC | |
| Prp-0-4 | #325 | CAA TGG AAC AAA CCC AGT AAG CCA AAA ACC AAC | |
| PrP-0-d5 | #325 | CAA TGG AAC AAA CCC AGT AAG CCA AAA ACC AAC | |
| PrP-0-d6 | #325 | CAA TGG AAC AAA CCC AGT AAG CCA AAA ACC AAC | |
| PrP-ARR | #325 | CAA TGG AAC AAA CCC AGT AAG CCA AAA ACC AAC | |
| | | Q W N K P S K P K T N | |
| bovine ex. | #358 | ATG AAG CAT GTG GCA GGA GCT GCT GCA GCT GGA | |
| | | M K H V A G A A A A G | |
| PrP-0-1 | #358 | ATG AAG CAT GTG GCA GGA GCT GCT GCA GCT GGA | |
| PrP-0-2 | #358 | ATG AAG CAT GTG GCA GGA GCT GCT GCA GCT GGA | |
| PrP-0-3 | #358 | ATG AAG CAT GTG GCA GGA GCT GCT GCA GCT GGA | |
| Prp-0-4 | #358 | ATG AAG CAT GTG GCA GGA GCT GCT GCA GCT GGA | |
| PrP-0-d5 | #358 | ATG AAG CAT GTG GCA GGA GCT GCT GCA GCT GGA | |
| PrP-O-d6 | #358 | ATG AAG CAT GTG GCA GGA GCT GCT GCA GCT GGA | |
| PrP-ARR | #358 | ATG AAG CAT GTG GCA GGA GCT GCT GCA GCT GGA | |
| | | M K H V A G A A A A G | |
| bovine ex. | #391 | GCA GTG GTA GGG GGC CTT GGT GGC TAC ATG CTG | |
| | | A V V G G L G G Y M L | |
| PrP-0-1 | #391 | GCA GTG GTA GGG GGC CTT GGT GGC TAC ATG CTG | |
| PrP-0-2 | #391 | GCA GTG GTA GGG GGC CTT GGT GGC TAC ATG CTG | |
| PrP-0-3 | #391 | GCA GTG GTA GGG GGC CTT GGT GGC TAC ATG CTG | |
| Prp-0-4 | #391 | GCA GTG GTA GGG GGC CTT GGT GGC TAC ATG CTG | |
| PrP-0-d5 | #391 | GCA GTG GTA GGG GGC CTT GGT GGC TAC ATG CTG | |
| PrP-0-d6 | #391 | GCA GTG GTA GGG GGC CTT GGT GGC TAC ATG CTG | |
| PrP-ARR | #391 | GCA GTG GTA GGG GGC CTT GGT GGC TAC ATG CTG | |
| | | A V V G G L G G Y M L | |
| bovine ex. | #424 | GGA AGT GCC ATG AGC AGG CCT CTT ATA CAT TTT | |
| | | G S A M S R P L I H F | |
| PrP-0-1 | #424 | GGA AGT GCC ATG AGC AGG CCT CTT ATA CAT TTT | |
| PrP-0-2 | #424 | GGA AGT GCC ATG AGC AGG CCT CTT ATA CAT TTT | |
| PrP-0-3 | #424 | GGA AGT GCC ATG AGC AGG CCT CTT ATA CAT TTT | |
| Prp-0-4 | #424 | GGA AGT GCC ATG AGC AGG CCT CTT ATA CAT TTT | |
| PrP-0-d5 | #424 | GGA AGT GCC ATG AGC AGG CCT CTT ATA CAT TTT | |
| PrP-0-d6 | #424 | GGA AGT GCC ATG AGC AGG CCT CTT ATA CAT TTT | |
| PrP-ARR | #424 | GGA AGT GCC ATG AGC AGG CCT CTT ATA CAT TTT | |
| | | G S A M S R P L I H F | |
| bovine ex. | #457 | GGC AGT GAC TAT GAG GAC CGT TAC TAT CGT GAA | |
| | | G S D Y E D R Y Y R E | |
| PrP-0-1 | #457 | GGC AGT GAC TAT GAG GAC CGT TAC TAT CGT GAA | |
| PrP-0-2 | #457 | GGC AGT GAC TAT GAG GAC CGT TAC TAT CGT GAA | |
| PrP-0-3 | #457 | GGC AGT GAC TAT GAG GAC CGT TAC TAT CGT GAA | |
| Prp-0-4 | #457 | GGC AGT GAC TAT GAG GAC CGT TAC TAT CGT GAA | |
| PrP-0-d5 | #457 | GGC AGT GAC TAT GAG GAC CGT TAC TAT CGT GAA | |
| PrP-0-d6 | #457 | GGC AGT GAC TAT GAG GAC CGT TAC TAT CGT GAA | |
| PrP-ARR | #457 | GGC AGT GAC TAT GAG GAC CGT TAG TAT CGT GAA | |
| | | G S D Y E D R Y Y R E | |
| bovine ex. | #490 | AAC ATG CAC CGT TAC CCC AAC CAA GTG TAC TAC | |
| | | N M H R Y P N Q V Y Y | |
| PrP-0-1 | #490 | AAC ATG CAC CGT TAC CCC AAC CAA GTG TAC TAC | |
| PrP-0-2 | #490 | AAC ATG CAC CGT TAC CCC AAC CAA GTG TAC TAC | |
| PrP-0-3 | #490 | AAC ATG CAC CGT TAC CCC AAC CAA GTG TAC TAC | |
| Prp-0-4 | #490 | AAC ATG CAC CGT TAC CCC AAC CAA GTG TAC TAC | |
| PrP-0-d5 | #490 | AAC ATG CAC CGT TAC CCC AAC CAA GTG TAC TAC | |
| PrP-0-d6 | #490 | AAC ATG CAC CGT TAC CCC AAC CAA GTG TAC TAC | |
| PrP-ARR | #490 | AAC ATG CAC CGT TAC CCC AAC CAA GTG TAC TAC | |
| | | N M H R Y P N Q V Y Y | |
| bovine ex. | #523 | AGG CCA GTG GAT CAG TAT AGT AAC CAG AAC AAC | |
| | | R P V D Q Y S N Q N N | |
| PrP-0-1 | #523 | AGG CCA GTG GAT CAG TAT AGT AAC CAG AAC AAC | |
| PrP-0-2 | #523 | AGG CCA GTG GAT CAG TAT AGT AAC CAG AAC AAC | |
| PrP-0-3 | #523 | AGG CCA GTG GAT CAG TAT AGT AAC CAG AAC AAC | |
| Prp-0-4 | #523 | AGG CCA GTG GAT CAG TAT AGT AAC CAG AAC AAC | |
| PrP-0-d5 | #523 | AGG CCA GTG GAT CAG TAT AGT AAC CAG AAC AAC | |
| PrP-0-d6 | #523 | AGG CCA GTG GAT CAG TAT AGT AAC CAG AAC AAC | |
| PrP-ARR | #523 | AGG CCA GTG GAT CGG TAT AGT AAC CAG AAC AAC | |
| | | R P V D R Y S N Q N N | |
| | | * AA171 | |
| bovine ex. | #556 | TTT GTG CAT GAC TGT GTC AAC ATC ACA GTC AAG | |
| | | F V H D C V N I T V K | |
| PrP-0-1 | #556 | TTT GTG CAT GAC TGT GTC AAC ATC ACA GTC AAG | |
| PrP-0-2 | #556 | TTT GTG CAT GAC TGT GTC AAC ATC ACA GTC AAG | |
| PrP-0-3 | #556 | TTT GTG CAT GAC TGT GTC AAC ATC ACA GTC AAG | |
| Prp-0-4 | #556 | TTT GTG CAT GAC TGT GTC AAC ATC ACA GTC AAG | |
| PrP-0-d5 | #556 | TTT GTG CAT GAC TGT GTC AAC ATC ACA GTC AAG | |
| PrP-0-d6 | #556 | TTT GTG CAT GAC TGT GTC AAC ATC ACA GTC AAG | |
| PrP-ARR | #556 | TTT GTG CAT GAC TGT GTC AAC ATC ACA GTC AAG | |
| | | F V H D C V N I T V K | |
| bovine ex. | #589 | GAA CAC ACA GTC ACC ACC ACC ACC AAG GGG GAG | |
| | | E H T V T T T T K G E | |
| PrP-0-1 | #589 | GAA CAC ACA GTC ACC ACC ACC ACC AAG GGG GAG | |
| PrP-0-2 | #589 | GAA CAC ACA GTC ACC ACC ACC ACC AAG GGG GAG | |
| PrP-0-3 | #589 | GAA CAC ACA GTC ACC ACC ACC ACC AAG GGG GAG | |
| Prp-0-4 | #589 | GAA CAC ACA GTC ACC ACC ACC ACC AAG GGG GAG | |
| PrP-0-d5 | #589 | GAA CAC ACA GTC ACC ACC ACC ACC AAG GGG GAG | |
| PrP-0-d6 | #589 | GAA CAC ACA GTC ACC ACC ACC ACC AAG GGG GAG | |
| PrP-ARR | #589 | GAA CAC ACA GTC ACC ACC ACC ACC AAG GGG GAG | |
| | | E H T V T T T T K G E | |
| bovine ex. | #622 | AAC TTC ACC GAA ACT GAC ATC AAG ATG ATG GAG | |
| | | N F T E T D I K M M H | |
| PrP-0-1 | #622 | AAC TTC ACC GAA ACT GAC ATC AAG ATG ATG GAG | |
| PrP-O-2 | #622 | AAC TTC ACC GAA ACT GAC ATC AAG ATG ATG GAG | |
| PrP-0-3 | #622 | AAC TTC ACC GAA ACT GAC ATC AAG ATG ATG GAG | |
| Prp-0-4 | #622 | AAC TTC ACC GAA ACT GAC ATC AAG ATG ATG GAG | |
| PrP-0-d5 | #622 | AAC TTC ACC GAA ACT GAC ATC AAG ATG ATG GAG | |
| PrP-0-d6 | #622 | AAC TTC ACC GAA ACT GAC ATC AAG ATG ATG GAG | |
| PrP-ARR | #622 | AAC TTC ACC GAA ACT GAC ATC AAG ATG ATG GAG | |
| | | N F T E T D I K M M H | |
| bovine ex. | #655 | CGA GTG GTG GAG CAA ATG TGC ATT ACC CAG TAC | |
| PrP-0-1 | #655 | CGA GTG GTG GAG CAA ATG TGC ATT ACC CAG TAC | |
| PrP-0-2 | #655 | CGA GTG GTG GAG CAA ATG TGC ATT ACC CAG TAC | |
| PrP-0-3 | #655 | CGA GTG GTG GAG CAA ATG TGC ATT ACC CAG TAC | |
| Prp-0-4 | #655 | CGA GTG GTG GAG CAA ATG TGC ATT ACC CAG TAC | |
| PrP-0-d5 | #655 | CGA GTG GTG GAG CAA ATG TGC ATT ACC CAG TAC | |
| PrP-0-d6 | #655 | CGA GTG GTG GAG CAA ATG TGC ATT ACC CAG TAC | |
| PrP-ARR | #655 | CGA GTG GTG GAG CAA ATG TGC ATT ACC CAG TAC | |
| | | R V V E Q M C I T Q Y | |
| bovine ex. | #688 | CAG AGA GAA TCC CAG GCT TAT TAC CAA CGA GGG | |
| | | Q R E S Q A Y Y Q R G | |
| PrP-0-1 | #688 | CAG AGA GAA TCC CAG GCT TAT TAC CAA CGA GGG | |
| PrP-0-2 | #688 | CAG AGA GAA TCC CAG GCT TAT TAC CAA CGA GGG | |
| PrP-0-3 | #688 | CAG AGA GAA TCC CAG GCT TAT TAC CAA CGA GGG | |
| Prp-0-4 | #688 | CAG AGA GAA TCC CAG GCT TAT TAC CAA CGA GGG | |
| PrP-0-d5 | #688 | CAG AGA GAA TCC CAG GCT TAT TAC CAA CGA GGG | |
| PrP-0-d6 | #688 | CAG AGA GAA TCC CAG GCT TAT TAC CAA CGA GGG | |
| PrP-ARR | #688 | CAG AGA GAA TCC CAG GCT TAT TAC CAA CGA GGG | |
| | | Q R E S Q A Y Y Q R G | |
| bovine ex. | #721 | GCA AGT GTG ATC CTC TTC TCT TCC CCT CCT GTG | |
| | | A S V I L F S S P P V | |
| PrP-0-1 | #721 | GCA AGT GTG ATC CTC TTC TCT TCC CCT CCT GTG | |
| PrP-0-2 | #721 | GCA AGT GTG ATC CTC TTC TCT TCC CCT CCT GTG | |
| PrP-0-3 | #721 | GCA AGT GTG ATC CTC TTC TCT TCC CCT CCT GTG | |
| Prp-0-4 | #721 | GCA AGT GTG ATC CTC TTC TCT TCC CCT CCT GTG | |
| PrP-0-d5 | #721 | GCA AGT GTG ATC CTC TTC TCT TCC CCT CCT GTG | |
| PrP-G-d6 | #721 | GCA AGT GTG ATC CTC TTC TCT TCC CCT CCT GTG | |
| PrP-ARR | #721 | GCA AGT GTG ATC CTC TTC TCT TCC CCT CCT GTG | |
| | | A S V I L F S S P P V | |
| bovine ex. | #754 | ATC CTC CTC ATC TCT TTC CTC ATT TTT CTC ATA | |
| | | I L L I S F L I F L I | |
| PrP-0-1 | #754 | ATC CTC CTC ATC TCT TTC CTC ATT TTT CTC ATA | |
| | | I L L I S F L I F L I | |
| PrP-0-2 | #754 | ATC CTC CTC ATC TCT TTC CTC ATT TTT CTC ATA | |
| PrP-0-3 | #754 | ATC CTC CTC ATC TCT TTC CTC ATT TTT CTC ATA | |
| Prp-0-4 | #754 | ATC CTC CTC ATC TCT TTC CTC ATT TTT CTC ATA | |
| PrP-0-d5 | #754 | ATC CTC CTC ATC TCT TTC CTC ATT TTT CTC ATA | |
| PrP-0-d6 | #754 | ATC CTC CTC ATC TCT TTC CTC ATT TTT CTC ATA | |
| PrP-ARR | #754 | ATC CTC CTC ATC TCT TTC CTC ATT TTT CTC ATA | |
| | | I L L I S F L I F L I | |
| bovine ex. | #787 | GTA GGA TAG GGG CAA CCT TCC TGT TTT CAT TAT | |
| | | V G * | |
| PrP-0-1 | #787 | GTA GGA TAG GGG CAA CCT TCC TGT TTT CAT TAT | |
| PrP-0-2 | #787 | GTA GGA TAG GGG CAA CCT TCC TGT TTT CAT TAT | |
| PrP-0-3 | #787 | GTA GGA TAG GGG CAA CCT TCC TGT TTT CAT TAT | |
| Prp-0-4 | #787 | GTA GGA TAG GGG CAA CCT TCC TGT TTT CAT TAT | |
| PrP-0-d5 | #787 | GTA GGA TAG GGG CAA CCT TCC TGT TTT CAT TAT | |
| PrP-0-d6 | #787 | GTA GGA TAG GGG CAA CCT TCC TGT TTT CAT TAT | |
| PrP-ARR | #787 | GTA GGA TAG GGG CAA CCT TCC TGT TTT CAT TAT | |
| | | V G * | |
| bovine ex. | #820 | CTT CTT AAT CTT TAC CAG GTT GGG GGA GGG AGT | |
| PrP-0-1 | #820 | CTT CTT AAT CTT TAC CAG GTT GGG GGA GGG AGT | |
| PrP-0-2 | #820 | CTT CTT AAT CTT TAC CAG GTT GGG GGA GGG AGT | |
| PrP-0-3 | #820 | CTT CTT AAT CTT TAC CAG GTT GGG GGA GGG AGT | |
| prp-0-4 | #820 | CTT CTT AAT CTT TAC CAG GTT GGG GGA GGG AGT | |
| PrP-0-d5 | #820 | CTT CTT AAT CTT TAC CAG GTT GGG GGA GGG AGT | |
| PrP-0-d6 | #820 | CTT CTT AAT CTT TAC CAG GTT GGG GGA GGC AGT | |
| PrP-ARR | #820 | CTT CTT AAT CTT TAC CAG GTT GGG GGA GGG AGT | |

We claim:

1. A method of making cattle resistant to bovine spongioform encephalopathy, comprising:
a) providing a modifying composition comprising a DNA fragment having a length of between 100 and 1000 bp and having essentially the sequence of the bovine PrP gene modified to prevent translation of the PrP protein;
b) introducing the modifying composition into a somatic cell from a cow or bull and culturing the cell to produce a cell having a modified PrP gene;
c) isolating said modified cell from cells having an unmodified PrP gene; and
d) transferring a nucleus from said isolated cell into a competent bovine germ-line cell and generating a founder cow or bull from the germ-line cell.

2. The method of claim 1, wherein the DNA fragment is single stranded and the modifying composition is substantially free of DNA complementary to the fragment.

3. The method of claim 2, wherein the length of the single stranded fragment is between 200 and 800 nt.

4. The method of making cattle resistant to bovine spongioform encephalopathy which comprises the method of claim 2, and the further step of interbreeding a founder cow and a founder bull.

5. The method of claim 4, wherein the length of the single stranded fragment is between 200 and 800 nt.

6. The method of claim 2, wherein the modified cell is isolated using coupled detection.

7. A composition comprising a single stranded DNA fragment having a length of between 100 and 1000 nt and having essentially the sequence of the bovine PrP gene modified to prevent translation of the PrP protein, wherein the composition is substantially free of DNA complementary to the fragmen.

8. The composition of claim 7, wherein the length of the single stranded fragment is between 400 and 800 nt.

9. A method of making cattle resistant to bovine spongioform encephalopathy, comprising:
a) providing a modifying composition comprising a DNA fragment having a length of between 100 and 1000 bp and having essentially the sequence of the bovine PrP gene modified to encode a dominant disease-resistant PrP protein;
b) introducing the modifying composition into a somatic cell from a cow or bull and culturing the cell to produce a cell having a modified PrP gene;
c) isolating said modified cell from cells having an unmodified PrP gene; and
d) transferring a nucleus from said isolated cell into a competent bovine germ-line cell and generating a founder cow or bull from the germ-line cell.

10. The method of claim 9, wherein the dominant disease-resistant PrP protein contains a glutamine-to-arginine substitution at amino acid 178.

11. The method of claim 9, wherein the DNA fragment is single stranded and the modifying composition is substantially free of DNA complementary to the fragment.

12. The method of claim 11, wherein the length of the single stranded fragment is between 200 and 800 nt.

13. The method of making cattle resistant to bovine spongioform encephalopathy which comprises the method of claim 11, and the further step of interbreeding a founder cow and a founder bull.

14. The method of claim 13, wherein the length of the single stranded fragment is between 200 and 800 nt.

15. The method of claim 11, wherein the modified cell is isolated using coupled detection.

16. A composition comprising a single stranded DNA fragment having a length of between 100 and 1000 nt and having essentially the sequence of the bovine PrP gene modified to encode a dominant disease-resistant PrP protein, wherein the composition is substantially free of DNA complementary to the fragment.

17. The composition of claim 16, wherein the length of the single stranded fragment is between 400 and 800 nt.

18. A method of testing for bovine spongioform encephalopathy resistance in the offspring of a cow and a bull comprising:
a) obtaining a nucleic acid sample from an offspring of a cow and a bull, wherein at least one parent carries a modified PrP gene that confers resistance to bovine spongioform encephalopathy; and
b) determining whether the modified PrP gene is present in the sample;

19. The method of claim 17, which further comprises determining whether the wild-type PrP gene is present in the sample.


SEQUENCE LISTING (32 sequences)

SEQ ID NO: 1 - DNA, 17 bp, Artificial Sequence
  forward primer
gtggccatgtggagtga

SEQ ID NO: 2 - DNA, 21 bp, Artificial Sequence
  reverse primer
cccaacctggtaaagattaag

SEQ ID NO: 3 - DNA, 21 bp, Artificial Sequence
  reverse complement of SEQ ID NO2
cttaatctttaccaggttggg

SEQ ID NO: 4 - DNA, 21 bp, Artificial Sequence
  forward primer
ctgtttatagctgatgccact

SEQ ID NO: 5 - DNA, 17 bp, Artificial Sequence
  reverse primer
acggttgcctccaggac

SEQ ID NO: 6 - DNA, 17 bp, Artificial Sequence
  reverse complement of SEQ ID NO5
gtcctggaggcaaccgt

SEQ ID NO: 7 - DNA, 19 bp, Artificial Sequence
  reverse primer
ggcttactgggtttgttcc

SEQ ID NO: 8 - DNA, 19 bp, Artificial Sequence
  reverse complement of SEQ ID NO7
ggaacaaacccagtaagcc

SEQ ID NO: 9 - DNA, 21 bp, Artificial Sequence
  reverse primer
ggcctgtagtacacttggttg

SEQ ID NO: 10 - DNA, 21 bp, Artificial Sequence
  reverse complement of SEQ ID NO9
caaccaagtgtactacaggcc

SEQ ID NO: 11 - DNA, 27 bp, Artificial Sequence
  W9stop primer
cacataggcagttagatcctggttctc

SEQ ID NO: 12 - DNA, 27 bp, Artificial Sequence
  W18stop primer
tttgtggccatgtagagtgacgtgggc

SEQ ID NO: 13 - DNA, 27 bp, Artificial Sequence
  C24stop primer
gacgtgggcctctgaaagaagcgacca

SEQ ID NO: 14 - DNA, 27 bp, Artificial Sequence
  K3stop
gtcatcatggtgtaaagccacataggc

SEQ ID NO: 15 - DNA, 26 bp, Artificial Sequence
  V2del primer
gtcatcatggtaaaagccacataggc

SEQ ID NO: 16 - DNA, 26 bp, Artificial Sequence
  H5del primer
ggtgaaaagccaataggcagttggat

SEQ ID NO: 17 - DNA, 27 bp, Artificial Sequence
  Q178R primer
aggccagtggatcggtatagtaaccag

SEQ ID NO: 18 - DNA, 1089 bp, Bos taurus
  CDS (238)..(1032)
ctaggaaacagagccaggaattattttaaggtcaactttgtccttagagaaggaagagttgtgttaacactttacctataattactttcgtgagatgtatggaatgtgaagaatatttatgacctagactgtttatagctgatgccactgctatgcagtcattatgctacagactttaagtgatttttacatgggcatatgatgctgacaccctctttattttgcagataagtcatcatggtgaaaagccacataggcagttggatcctggttctctttgtggccatgtggagtgacgtgggcctctgcaagaagcgaccaaaacctggaggaggatggaacactggggggagccgatacccaggacagggcagtcctggaggcaaccgttatccacctcagggagggggtggctggggtcagccccatggaggtggctggggccagcctcatggaggtggctggggccagcctcatggaggtggctggggtcagccccatggtggtggctggggacagccacatggtggtggaggctggggtcaaggtggtacccacggtcaatggaacaaacccagtaagccaaaaaccaacatgaagcatgtggcaggagctgctgcagctggagcagtggtagggggccttggtggctacatgctgggaagtgccatgagcaggcctcttatacattttggcagtgactatgaggaccgttactatcgtgaaaacatgcaccgttaccccaaccaagtgtactacaggccagtggatcagtatagtaaccagaacaactttgtgcatgactgtgtcaatatcacagtcaaggaacacacagtcaccaccaccaccaagggggagaacttcaccgaaactgacatcaagatgatgaagcgagtggtggagcaaatgtgcattacccagtaccagagagaatcccaggcttattaccaacgaggggcaagtgtgatcctcttctcttcccctcctgtgatcctcctcatctctttcctcatttttctcatagtaggataggggcaaccttcctgttttcattatcttcttaatctttaccaggttgggggagggagt

SEQ ID NO: 19 - protein, 264 aa, Bos taurus
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGSDYEDRYYRENMHRYPNQVYYRPVDQYSNQNNFVHDCVNITVKEHTVTTTTKGENFTETDIKMMKRVVEQMCITQYQRESQAYYQRGASVILFSSPPVILLISFLIFLIVG

SEQ ID NO: 20 - DNA, 858 bp, Bos taurus
  CDS (7)..(33)
gtcatcatggtgaaaagccacataggcagttagatcctggttctctttgtggccatgtggagtgacgtgggcctctgcaagaagcgaccaaaacctggaggaggatggaacactggggggagccgatacccaggacagggcagtcctggaggcaaccgttatccacctcagggagggggtggctggggtcagccccatggaggtggctggggccagcctcatggaggtggctggggccagcctcatggaggtggctggggtcagccccatggtggtggctggggacagccacatggtggtggaggctggggtcaaggtggtacccacggtcaatggaacaaacccagtaagccaaaaaccaacatgaagcatgtggcaggagctgctgcagctggagcagtggtagggggccttggtggctacatgctgggaagtgccatgagcaggcctcttatacattttggcagtgactatgaggaccgttactatcgtgaaaacatgcaccgttaccccaaccaagtgtactacaggccagtggatcagtatagtaaccagaacaactttgtgcatgactgtgtcaatatcacagtcaaggaacacacagtcaccaccaccaccaagggggagaacttcaccgaaactgacatcaagatgatgaagcgagtggtggagcaaatgtgcattacccagtaccagagagaatcccaggcttattaccaacgaggggcaagtgtgatcctcttctcttcccctcctgtgatcctcctcatctctttcctcatttttctcatagtaggataggggcaaccttcctgttttcattatcttcttaatctttaccaggttgggggagggagt

SEQ ID NO: 21 - protein, 8 aa, Bos taurus
MVKSHIGS

SEQ ID NO: 22 - DNA, 858 bp, Bos taurus
  CDS (7)..(60)
gtcatcatggtgaaaagccacataggcagttggatcctggttctctttgtggccatgtagagtgacgtgggcctctgcaagaagcgaccaaaacctggaggaggatggaacactggggggagccgatacccaggacagggcagtcctggaggcaaccgttatccacctcagggagggggtggctggggtcagccccatggaggtggctggggccagcctcatggaggtggctggggccagcctcatggaggtggctggggtcagccccatggtggtggctggggacagccacatggtggtggaggctggggtcaaggtggtacccacggtcaatggaacaaacccagtaagccaaaaaccaacatgaagcatgtggcaggagctgctgcagctggagcagtggtagggggccttggtggctacatgctgggaagtgccatgagcaggcctcttatacattttggcagtgactatgaggaccgttactatcgtgaaaacatgcaccgttaccccaaccaagtgtactacaggccagtggatcagtatagtaaccagaacaactttgtgcatgactgtgtcaatatcacagtcaaggaacacacagtcaccaccaccaccaagggggagaacttcaccgaaactgacatcaagatgatgaagcgagtggtggagcaaatgtgcattacccagtaccagagagaatcccaggcttattaccaacgaggggcaagtgtgatcctcttctcttcccctcctgtgatcctcctcatctctttcctcatttttctcatagtaggataggggcaaccttcctgttttcattatcttcttaatctttaccaggttgggggagggagt

SEQ ID NO: 23 - protein, 17 aa, Bos taurus
MVKSHIGSWILVLFVAM

SEQ ID NO: 24 - DNA, 858 bp, Bos taurus
  CDS (7)..(78)
gtcatcatggtgaaaagccacataggcagttggatcctggttctctttgtggccatgtggagtgacgtgggcctctgaaagaagcgaccaaaacctggaggaggatggaacactggggggagccgatacccaggacagggcagtcctggaggcaaccgttatccacctcagggagggggtggctggggtcagccccatggaggtggctggggccagcctcatggaggtggctggggccagcctcatggaggtggctggggtcagccccatggtggtggctggggacagccacatggtggtggaggctggggtcaaggtggtacccacggtcaatggaacaaacccagtaagccaaaaaccaacatgaagcatgtggcaggagctgctgcagctggagcagtggtagggggccttggtggctacatgctgggaagtgccatgagcaggcctcttatacattttggcagtgactatgaggaccgttactatcgtgaaaacatgcaccgttaccccaaccaagtgtactacaggccagtggatcagtatagtaaccagaacaactttgtgcatgactgtgtcaatatcacagtcaaggaacacacagtcaccaccaccaccaagggggagaacttcaccgaaactgacatcaagatgatgaagcgagtggtggagcaaatgtgcattacccagtaccagagagaatcccaggcttattaccaacgaggggcaagtgtgatcctcttctcttcccctcctgtgatcctcctcatctctttcctcatttttctcatagtaggataggggcaaccttcctgttttcattatcttcttaatctttaccaggttgggggagggagt

SEQ ID NO: 25 - protein, 23 aa, Bos taurus
MVKSHIGSWILVLFVAMWSDVGL

SEQ ID NO: 26 - DNA, 858 bp, Bos taurus
  CDS (7)..(15)
gtcatcatggtgtaaagccacataggcagttggatcctggttctctttgtggccatgtggagtgacgtgggcctctgcaagaagcgaccaaaacctggaggaggatggaacactggggggagccgatacccaggacagggcagtcctggaggcaaccgttatccacctcagggagggggtggctggggtcagccccatggaggtggctggggccagcctcatggaggtggctggggccagcctcatggaggtggctggggtcagccccatggtggtggctggggacagccacatggtggtggaggctggggtcaaggtggtacccacggtcaatggaacaaacccagtaagccaaaaaccaacatgaagcatgtggcaggagctgctgcagctggagcagtggtagggggccttggtggctacatgctgggaagtgccatgagcaggcctcttatacattttggcagtgactatgaggaccgttactatcgtgaaaacatgcaccgttaccccaaccaagtgtactacaggccagtggatcagtatagtaaccagaacaactttgtgcatgactgtgtcaatatcacagtcaaggaacacacagtcaccaccaccaccaagggggagaacttcaccgaaactgacatcaagatgatgaagcgagtggtggagcaaatgtgcattacccagtaccagagagaatcccaggcttattaccaacgaggggcaagtgtgatcctcttctcttcccctcctgtgatcctcctcatctctttcctcatttttctcatagtaggataggggcaaccttcctgttttcattatcttcttaatctttaccaggttgggggagggagt

SEQ ID NO: 27 - DNA, 857 bp, Bos taurus
  CDS (7)..(24)
gtcatcatggtaaaagccacataggcagttggatcctggttctctttgtggccatgtggagtgacgtgggcctctgcaagaagcgaccaaaacctggaggaggatggaacactggggggagccgatacccaggacagggcagtcctggaggcaaccgttatccacctcagggagggggtggctggggtcagccccatggaggtggctggggccagcctcatggaggtggctggggccagcctcatggaggtggctggggtcagccccatggtggtggctggggacagccacatggtggtggaggctggggtcaaggtggtacccacggtcaatggaacaaacccagtaagccaaaaaccaacatgaagcatgtggcaggagctgctgcagctggagcagtggtagggggccttggtggctacatgctgggaagtgccatgagcaggcctcttatacattttggcagtgactatgaggaccgttactatcgtgaaaacatgcaccgttaccccaaccaagtgtactacaggccagtggatcagtatagtaaccagaacaactttgtgcatgactgtgtcaatatcacagtcaaggaacacacagtcaccaccaccaccaagggggagaacttcaccgaaactgacatcaagatgatgaagcgagtggtggagcaaatgtgcattacccagtaccagagagaatcccaggcttattaccaacgaggggcaagtgtgatcctcttctcttcccctcctgtgatcctcctcatctctttcctcatttttctcatagtaggataggggcaaccttcctgttttcattatcttcttaatctttaccaggttgggggagggagt

SEQ ID NO: 28 - protein, 5 aa, Bos taurus
MVKAT

SEQ ID NO: 29 - DNA, 857 bp, Bos taurus
  CDS (7)..(24)
gtcatcatggtgaaaagccaataggcagttggatcctggttctctttgtggccatgtggagtgacgtgggcctctgcaagaagcgaccaaaacctggaggaggatggaacactggggggagccgatacccaggacagggcagtcctggaggcaaccgttatccacctcagggagggggtggctggggtcagccccatggaggtggctggggccagcctcatggaggtggctggggccagcctcatggaggtggctggggtcagccccatggtggtggctggggacagccacatggtggtggaggctggggtcaaggtggtacccacggtcaatggaacaaacccagtaagccaaaaaccaacatgaagcatgtggcaggagctgctgcagctggagcagtggtagggggccttggtggctacatgctgggaagtgccatgagcaggcctcttatacattttggcagtgactatgaggaccgttactatcgtgaaaacatgcaccgttaccccaaccaagtgtactacaggccagtggatcagtatagtaaccagaacaactttgtgcatgactgtgtcaatatcacagtcaaggaacacacagtcaccaccaccaccaagggggagaacttcaccgaaactgacatcaagatgatgaagcgagtggtggagcaaatgtgcattacccagtaccagagagaatcccaggcttattaccaacgaggggcaagtgtgatcctcttctcttcccctcctgtgatcctcctcatctctttcctcatttttctcatagtaggataggggcaaccttcctgttttcattatcttcttaatctttaccaggttgggggagggagt

SEQ ID NO: 30 - protein, 5 aa, Bos taurus
MVKSQ

SEQ ID NO: 31 - DNA, 858 bp, Bos taurus
  CDS (7)..(801)
gtcatcatggtgaaaagccacataggcagttggatcctggttctctttgtggccatgtggagtgacgtgggcctctgcaagaagcgaccaaaacctggaggaggatggaacactggggggagccgatacccaggacagggcagtcctggaggcaaccgttatccacctcagggagggggtggctggggtcagccccatggaggtggctggggccagcctcatggaggtggctggggccagcctcatggaggtggctggggtcagccccatggtggtggctggggacagccacatggtggtggaggctggggtcaaggtggtacccacggtcaatggaacaaacccagtaagccaaaaaccaacatgaagcatgtggcaggagctgctgcagctggagcagtggtagggggccttggtggctacatgctgggaagtgccatgagcaggcctcttatacattttggcagtgactatgaggaccgttactatcgtgaaaacatgcaccgttaccccaaccaagtgtactacaggccagtggatcggtatagtaaccagaacaactttgtgcatgactgtgtcaatatcacagtcaaggaacacacagtcaccaccaccaccaagggggagaacttcaccgaaactgacatcaagatgatgaagcgagtggtggagcaaatgtgcattacccagtaccagagagaatcccaggcttattaccaacgaggggcaagtgtgatcctcttctcttcccctcctgtgatcctcctcatctctttcctcatttttctcatagtaggataggggcaaccttcctgttttcattatcttcttaatctttaccaggttgggggagggagt

SEQ ID NO: 32 - protein, 264 aa, Bos taurus
MVKSHIGSWILVLFVAMWSDVGLCKKRPKPGGGWNTGGSRYPGQGSPGGNRYPPQGGGGWGQPHGGGWGQPHGGGWGQPHGGGWGQPHGGGWGQPHGGGGWGQGGTHGQWNKPSKPKTNMKHVAGAAAAGAVVGGLGGYMLGSAMSRPLIHFGSDYEDRYYRENMHRYPNQVYYRPVDRYSNQNNFVHDCVNITVKEHTVTTTTKGENFTETDIKMMKRVVEQMCITQYQRESQAYYQRGASVILFSSPPVILLISFLIFLIVG